(19) United States
(12) Patent Application Publication
(10) Pub. No.: US 2026/0102543 A1
(43) Pub. Date: Apr. 16, 2026

(54) CATHETER LINERS, ASSEMBLIES, AND METHODS FOR PRODUCING SAME

(71) Applicant: Zeus Company LLC, ORANGEBURG, SC (US)

(72) Inventors: H. Edward Rast, III, Cameron, SC (US); Morgan Baltzegar, St. Matthews, SC (US); Kenneth David Gray, JR., Columbia, SC (US); Alexandra Younan, Columbia, SC (US); Christopher G. Fassuliotis, West Columbia, SC (US); Dana Barringer, Orangeburg, SC (US)

(21) Appl. No.: 18/915,706

(22) Filed: Oct. 15, 2024

(51) Int. Cl. A61L 29/04; A61L 29/12; A61M 25/00

(52) U.S. Cl. CPC A61L 29/041; A61L 29/126; A61M 25/0045; A61M 25/005; A61L 2420/04

(57) ABSTRACT

The present disclosure provides catheters including non-fluorinated liners comprising ultra-high molecular weight polyethylene (UHMWPE) with advantageous combinations of strength, flexibility, and size. The catheters may further include an optional reinforcement layer over the tube with either stainless steel or polymeric braids and a jacketing material to cover the braid and adhere to the liner, providing flexibility and lubricity to the outer diameter of the catheter assembly.

### FIELD

The present application relates generally to the field of catheters and to methods relating to such catheters for use in minimally invasive medical applications.

### BACKGROUND

Certain medical applications use catheter-based procedures and specialized equipment and techniques. Catheters used in these procedures commonly comprise several components including a coating or liner on the inner wall to provide a smooth inner surface; a reinforcement layer over the inner liner and a jacketing layer to adhere to the braid and liner.

The liner is the innermost layer of a catheter shaft, providing a smooth, low-friction surface that facilitates the movement of fluids, guidewires, stents, and other devices. Among the various materials that have been pursued as an inner liner, polytetrafluoroethylene (PTFE) is foremost due to its excellent chemical resistance (e.g., to corrosive chemicals), high temperature resistance, excellent biocompatibility, high impact strength, excellent wear resistance, very low coefficient of friction/high lubricity, and low cost. It has been used in joint implants for over 40 years, particularly as an articular liner in total hip replacements and tibial insert in total knee replacements.

One major drawback of PTFE liners is that it is not radiation-stable. Radiation sterilization (i.e., gamma rays or electron beams) is one of the most widely used and safe sterilization processes for medical devices, but is not applicable for PTFE liners. Radiation sterilization improves the manufacturability of catheters as it can be quickly performed in the manufacturing line, while the ethylene oxide gas sterilization (ETO) procedure, typically used with PTFE-lined catheters, requires that the sterilized PTFE be stored for up to 48 hours to allow the gas to diffuse out of the sterilized equipment. Also, ethylene oxide gas requires careful handling because of its flammability and toxicity. Strict handling requirements and a technically complex sterilization process makes ETO sterilization technique often undesirable. In recent times, medical regulatory organizations worldwide have also been encouraging the medical industry to minimize or replace the use of ETO with alternative sterilization methods. Another shortcoming of PTFE liners is the difficulty encountered in adhering the liner to other components of a catheter, e.g., a reinforcement layer and/or a jacket. PTFE must be surface-treated to promote adhesion to these additional components, and subsequent handling of PTFE liners during catheter construction operations can reduce the effectiveness of the surface treatment in providing adequate adhesion, which can lead to delamination of the layers in use.

While several other polymers can advantageously withstand gamma irradiation, none can match certain advantageous physical features of PTFE, such as the lubricity and/or low coefficient of friction of PTFE. As such, alternative liners would be desirable to address the concerns with conventional PTFE liners, while still exhibiting desirable physical properties rendering them suitable for use as catheter liners.

### SUMMARY

The disclosure provides UHMWPE liners, catheters comprising such liners (e.g., further comprising an optional reinforcement layer and a jacket), and methods of making and using such liners and catheters. In some embodiments, the disclosed UHMWPE can be considered to be replacements for conventional PTFE liners for a range of applications. PTFE liners have a unique combination of high tensile strength and high flexibility not typically found in polyethylene. Ultra-High Molecular Weight Polyethylene (“UHMWPE”), with its higher molecular weight as compared with HDPE (“High Density Polyethylene”) would be expected to exhibit a correspondingly higher tensile strength then HDPE when formed into tubes and thus might be expected to be a more suitable replacement for PTFE than HDPE. However, t has been generally understood that, while UHMWPE can be comparable to PTFE in terms of molecular weight and thus may provide a reasonable tensile strength, the stiffness associated with UHMWPE increases as the molecular weight increases, thereby adversely impacting the flexibility of the material (which is a desirable feature of catheter liners).

The inventors have surprisingly found that when the disclosed UHMWPE liners are used as catheter components as described herein, the catheter properties are similar to the properties of PTFE-lined catheters in simulated end-use. While similar liners have been disclosed, prior art does not teach the utility of these liners in a catheter where trackability is optimized to simulate end-use in a vasculature. The properties of the liner of Singhi cannot be used to infer behavior in a finished catheter assembly that has to track through a vasculature. The final construction of a catheter whether braided or coiled has inherently unique characteristics that affect the trackability. This invention quantifies the interaction of those unique components in a catheter assembly enabling optimization of the components used for specific end-uses.

In some embodiments, catheters disclosed herein comprise an UHMWPE liner that has been formed by a dip-coating process such as described in US 2024/0066186 to Singhi, which is incorporated herein by reference in its entirety.

In some embodiments, catheters disclosed herein comprise an UHMWPE liner that has been formed in a paste extrusion process such as described in US 2024/0066777 To Singhi, which is incorporated herein by reference in its entirety.

In some embodiments, catheters disclosed herein comprise an UHMWPE liner that has been formed in molding processes that are known in the art. Such methods can include, but are not limited to, for example, wrapping a mandrel with UHMWPE sheet to form a tube, or wrapping UHMWPE membrane or wrapping expanded-UHMWPE sheet.

In some embodiments, the UHMWPE liner comprises a coating on at least a portion of its outer surface, wherein the coating comprises maleic anhydride grafted polyethylene. In some embodiments, the UHMWPE liner comprises a coating on at least a portion of its outer surface, wherein the coating comprises a maleic anhydride grafted copolymer of ethylene such as, but not limited to, ethylene vinyl acetate.

In some embodiments, catheters disclosed herein comprise a reinforcement layer comprising a wire or polymeric braid. In some embodiments, catheters disclosed herein comprise a reinforcement layer comprising a wire or polymeric coil. In some embodiments, catheters disclosed herein comprise a reinforcement layer comprising both braids and coils.

In some embodiments, catheters disclosed herein comprise a reinforcement layer that does not extend the entire length of the catheter. For example, the reinforcement layer may extend about 25% or more, about 50% or more, about 75% or more, e.g., about 25% to about 100%, about 25% to about 75%, about 25% to about 50%, about 50% to about 75%, about 50% to about 100%, or about 75% to about 100% the length of the catheter or the length of the liner. In some embodiments, the catheters of the current invention comprise no reinforcement layer.

In some embodiments, catheters disclosed herein comprise an outer jacket, comprising one or more grades of polyamide. In some embodiments, catheters disclosed herein comprise an outer jacket comprising one or more grades of polyurethane. In some embodiments, catheters disclosed herein comprise an outer jacket comprising one or more grades of polyamide, polyurethane, and/or PEBA. In some embodiments, catheters disclosed herein comprise an outer jacket comprising one or more grades of PEBA.

In some embodiments, catheters disclosed herein comprise an outer jacket that does not extend the entire length of the catheter. For example, the outer jacket may extend about 25% or more, about 50% or more, about 75% or more, e.g., about 25% to about 100%, about 25% to about 75%, about 25% to about 50%, about 50% to about 75%, about 50% to about 100%, or about 75% to about 100% the length of the catheter or the length of the liner.

The disclosure includes, without limitation, the following embodiments.
Embodiment 1: A catheter comprising an Ultra High Molecular Weight Poly(Ethylene) (“UHMWPE”) liner, an optional reinforcement layer, and a jacket, wherein the catheter comprises one or both of: a) a Maximum Work of Advancement of 12,000 mN·cm or less; and b) a Maximum Work of Advancement of a 0.014″ guidewire of 2500 mN·cm or less.
Embodiment 2: The catheter of Embodiment 1, further comprising a tie layer overlying at least a portion of the UHMWPE liner.
Embodiment 3: The catheter of Embodiment 2, wherein the tie layer comprises a functionalized polyethylene.
Embodiment 4: The catheter of Embodiment 3, wherein the functionalized polyethylene comprises maleic anhydride grafted polyethylene.
Embodiment 5: The catheter of any of Embodiments 1-4, comprising the reinforcement layer.
Embodiment 6: The catheter of Embodiment 5, wherein the reinforcement layer is selected from the group consisting of a coil, a braid, or a combination thereof.
Embodiment 7: The catheter of Embodiment 5, wherein the reinforcement layer comprises a metallic or polymeric material.
Embodiment 8: The catheter of any of Embodiments 1-7, wherein the UHMWPE liner comprises UHMWPE comprising at least 85% ethylene-derived monomer units.
Embodiment 9: The catheter liner of Embodiment 8, wherein the UHMWPE further comprises one or more α-olefins.
Embodiment 10: The catheter liner of Embodiment 9, wherein the one or more α-olefins are selected from the group consisting of 1-butene, 1-pentene, 1-hexene, 1-octene, 1-decene, 1-dodecene, 4-methyl-1-pentene, 3-methyl-1-pentene, and combinations thereof.
Embodiment 11: The catheter of any of Embodiments 1-10, wherein the UHMWPE liner consists essentially of UHMWPE.
Embodiment 12: The catheter of any of Embodiments 1-10, wherein the UHMWPE liner comprises one or more particulate fillers.
Embodiment 13: The catheter of any of Embodiments 1-10, wherein the UHMWPE liner consists essentially of UHMWPE and one or more particulate fillers.
Embodiment 14: The catheter of any of Embodiments 1-13, wherein the catheter exhibits a Maximum Force of Advancement of a 0.014″ guidewire that is at least about 20% lower than a Maximum Force of Advancement of a 0.014″ guidewire of a comparable HDPE-lined catheter.
Embodiment 15: The catheter of any of Embodiments 1-14, wherein the catheter exhibits a Work of Advancement of a 0.014″ guidewire that is at least about 25% lower than a Work of Advancement of a 0.014″ guidewire of a comparable HDPE-lined catheter.
Embodiment 16: The catheter of any of Embodiments 1-15, wherein the catheter exhibits a Work of Advancement of a 0.014″ guidewire that is within about 20% of a Work of Advancement of a 0.014″ guidewire of a comparable PTFE-lined catheter.
Embodiment 17: The catheter of any of Embodiments 1-16, wherein the catheter exhibits a maximum Force of Advancement of 1200 mN or less.
Embodiment 18: The catheter of any of Embodiments 1-17, wherein the catheter exhibits a maximum Work of Advancement of 12,000 mN·cm or less.
Embodiment 19: The catheter of any of Embodiments 1-18, wherein the catheter exhibits a maximum Force of Advancement of a 0.014″ guidewire of 60 mN or less.
Embodiment 20: The catheter of any of Embodiments 1-19, wherein the catheter exhibits a maximum Work of Advancement of a 0.014″ guidewire of 2500 mN·cm or less.

These and other features, aspects, and advantages of the disclosure will be apparent from a reading of the following detailed description, together with the accompanying drawings, which are briefly described below. The invention includes any combination of two, three, four, or more of the above-noted embodiments, as well as combinations of any two, three, four, or more features or elements set forth in this disclosure, regardless of whether such features or elements are expressly combined in a specific embodiment description herein. This disclosure is intended to be read holistically such that any separate features or elements of the disclosed invention, in any of its various aspects and embodiments, should be viewed as intended to be combinable unless the context clearly dictates otherwise.

### BRIEF DESCRIPTION OF THE DRAWINGS

In order to provide an understanding of the embodiments of the invention, reference is made to the appended drawings, which are not necessarily drawn to scale, and in which reference numerals refer to components of exemplary embodiments of the invention. The drawings are exemplary only, and should not be construed as limiting the invention.

FIG. 1 is a longitudinal cross-sectional view of a non-limiting catheter shaft according to certain embodiments of the present disclosure with jacket 120 removed from the distal end to show braided reinforcement layer 110 and inner liner 100.

FIG. 2 is a longitudinal cross-sectional view of a non-limiting catheter shaft according to certain embodiments of the present disclosure with jacket 120 removed from the distal end to show coiled reinforcement layer 130 and inner liner 100.

FIG. 3 is a schematic diagram of the S-curve test track used with the IDTE 3000 showing all dimensions in inches. The proximal end of the track is at the bottom of the figure.

FIG. 4 shows a force versus distance curve within the test track showing Maximum Force and Work of Insertion.

### DETAILED DESCRIPTION

The present disclosure provides ultra-high molecular weight (UHMWPE) catheter liners, catheter assemblies comprising such catheter liners, and methods of making and using such catheter liners and catheter assemblies. Advantageously, by developing catheter assemblies comprising UHMWPE catheter liners, the inventors have found that catheter assemblies can be provided which exhibit excellent physical properties previously not understood to be achievable using catheter liners comprising non-fluorinated materials.

The present disclosure will now be described more fully hereinafter with reference to example embodiments thereof. These example embodiments are described so that this disclosure will be thorough and complete, and will fully convey the scope of the disclosure to those skilled in the art. Indeed, the disclosure may be embodied in many different forms and should not be construed as limited to the embodiments set forth herein; rather, these embodiments are provided so that this disclosure will satisfy applicable legal requirements.

As used in this specification and the claims, the singular forms “a,” “an,” and “the” include plural referents unless the context clearly dictates otherwise. Reference to “dry weight percent” or “dry weight basis” refers to weight on the basis of dry ingredients (i.e., all ingredients except water). Reference to “wet weight” refers to the weight of the mixture including water. Unless otherwise indicated, reference to “weight percent” of a mixture reflects the total wet weight of the mixture (i.e., including water).

### Catheter Liners

A liner as provided herein is generally a tubular form, which is generally cylindrical in shape. Liners of various sizes are known and are intended to be encompassed by the present disclosure. For example, liners are generally defined by features such as their length, outer diameter (OD), inner diameter (ID), and wall thickness (where OD-ID=wall thickness). In some embodiments, the disclosed liners have an average wall thickness of 0.004″ or less or 0.002″ or less, e.g., about 0.0001″ to about 0.004″, such as about 0.0002″ to about 0.001″, about 0.0002″ to about 0.002″, about 0.0002 to about 0.003″, or about 0.0005″ to about 0.001.″ In some embodiments, the disclosed liners have IDs ranging from 0.013″ to 0.400″. The length of a given liner can be any length suitable for use e.g., in a catheter construction. In some non-limiting embodiments, the length 1 is about 6″ to about 20″, such as about 12″ to about 20″.

The liners described herein are referenced as being “ultra-high molecular weight polyethylene” (“UHMWPE”) liners. By “UHMWPE” liners is meant that at least a portion of the liner comprises UHMWPE. In some embodiments, the liners comprise at least 75%, at least 80%, at least about 85%, at least about 90%, at least about 95%, at least about 98%, at least about 99%, or 100% UHMWPE. In some embodiments, the disclosed liners comprise, consist essentially of, or consist of UHMWPE.

Where other components are present within the disclosed liners, they may be, for example, fillers and/or other polymers. In some embodiments, the tubes consist essentially of UHMWPE in combination with another polymer and/or a filler. In some embodiments, additional polymers can be included within the liners to impart specific properties such as lubrication, toughness or adhesion. For example, in some embodiments, the liners comprise a polymer that is a tie resin, such as a polyethylene based tie-resin (e.g., including, but not limited to, low-density polyethylene (LDPE), high density polyethylene (HDPE), linear low-density polyethylene (LLDPE), very low density polyethylene (VLDPE), and derivatives, copolymers, and combinations thereof). Further suitable polyethylene-based tie resins include, but are not limited to, anhydride modified polyethylene, ethylene vinyl acetate, ethylene methyl acrylate, ethylene acrylic acid, ethylene methacrylic acid, ethylene-acrylic ester-maleic anhydride terpolymer, and the like. Where liners are provided that comprise fillers, the fillers can be particulate fillers and the fillers can be incorporated to impart specific properties to the liner, such as color, radiopacity, strength and/or hydrophilicity. Liners with added fillers and/or polymers other than UHMWPE may have different properties than liners consisting essentially of UHMWPE alone, such as different mechanical, thermal and barrier properties, different crystallinities, different coefficients of friction, etc.

The UHMWPE of the liners is generally a polyethylene-based polymer that comprises primarily ethylene-derived units (with a repeating unit of —CH₂—CH₂). In some embodiments, the UHMWPE of the liners comprises at least 50%, at least 55%, at least 60%, at least 65%, at least 70%, at least 75%, at least 80%, at least 85%, at least 90%, at least 95%, at least 96%, at least 97%, at least 98%, at least 99%, at least 99.9%, or 100% ethylene-derived units. In some embodiments, the UHMWPE is a homopolymer of ethylene. In other embodiments, the UHMWPE is a copolymer of ethylene and an α-olefin such as 1-butene, 1-pentene, 1-hexene, 1-octene, 1-decene, 1-dodecene, 4-methyl-1-pentene, or 3-methyl-1-pentene. Exemplary resins that can be processed to provide liners of the present disclosure include, but are not limited to, Celanese's [GUR® 2024, GUR® 2122, GUR® 2122-5, GUR® 2126, GUR® 4012, GUR® 4012 F, GUR® 4020-3, GUR® 4022, GUR® 4022-6, GUR® 4032, GUR® 4050-3, GUR® 4056-3, GUR® 4112, GUR® 4113, GUR® 4120, GUR® 4122, GUR® 4122-5, GUR® 4130, GUR® 4150, GUR® 4150-3, GUR® 4152, GUR® 4170, GUR® 4523, GUR® 4550, GUR® 5113, GUR®5129, GUR® 5523, GUR® X161, GUR® X 195, GUR® X204, GUR® X 214, GUR® X217], Mitsui's [Mipelon PM200, XM220, XM221U, XM330], Hi-Zex Million 030S, 145M, 240S, 320MU, 630M, Braskem's UTEC3040, UTEC3041, UTEC4040, UTEC4041, UTEC5540, UTEC5541, UTEC6540, UTEC6540G, UTEC6541, Rochling's Polystone, LyondellBasell's Lupolen UHM 5000, and Asahi Kasei's Sunfine UH. In some embodiments, medical grade UHMWPE is used, which has a molecular mass greater than 1×10⁶ g/mol and is a semi-crystalline polymer.

Various polymers classified as “UHMWPE” are known and can be used according to the present disclosure. The UHMWPE in some embodiments has a weight average molecular weight (Mw) of greater than 1,000,000 g/mol, greater than 1,500,000 g/mol, or greater than 1,750,000 g/mol, or greater than 1,850,000 g/mol, or greater than 1,900,000 g/mol. For example, in some embodiments, the Mw of the UHMWPE is 1,000,000 g/mol to 3,000,000 g/mol, e.g., 1,000,000 g/mol to 2,500,000 g/mol, 1,500,000 g/mol to 3,000,000 g/mol, e.g., 1,500,000 g/mol to 2,500,000 g/mol, 1,750,000 g/mol to 3,000,000 g/mol, 1,750,000 g/mol to 2,500,000 g/mol, 1,850,000 g/mol to 3,000,000 g/mol, 1,850,000 g/mol to 2,500,000 g/mol 1,900,000 g/mol to 3,000,000 g/mol, or 1,900,000 g/mol to 2,500,000 g/mol. UHMWPE has very low coefficient of friction, excellent wear resistance, good toughness, high impact strength, high resistance to corrosive chemicals, excellent biocompatibility, and low cost. It has been used in joint implants for over 40 years, particularly as an articular liner in total hip replacements and tibial insert in total knee replacements.

The UHMWPE in some embodiments has a density greater than 0.91 g/cc or greater than 0.92 g/cc, or greater than 0.93 g/cc. In some embodiments, the UHMWPE has a density of from 0.91 to 0.96 g/cc, or from 0.92 to 0.95 g/cc.

The UHMWPE in some embodiments has a Shore D hardness of greater than 50, or greater than 55, or greater than 57, or greater than 60 (ASTM D2240). The UHMWPE may have a Shore D hardness of less than 100, or less than 90, or less than 80, or less than 75.

The UHMWPE may have a melting point (ASTM D3418) of greater than 100° C., or greater than 110° C., or greater than 115° C., or greater than 120° C., or greater than 125° C., or greater than 130° C. The UHMWPE may have a melting point of less than 200° C., or less than 190° C., or less than 180° C., or less than 170° C., or less than 160° C., or less than 150° C., or less than 140° C.

U.S. Pat. No. 8,308,711 discloses UHMWPE as a liner material, but the melt flow rate (“MFR”) is specified to be greater than 5.0 in order for the material to be processed by melt extrusion. The UHMWPE of the liners provided herein have molecular weights exceeding 1,000,000 g/mol, with exemplary melt flow rates (MFR) at 190° C. and 21.6 kg of less than 1.0 g/10 min and cannot be processed by melt extrusion.

Useful UHMWPE liners in the context of the present disclosure can, in some embodiments, be characterized by parameters such as stress and strain at break, tensile and storage modulus values, and coefficient of friction. In some embodiments, suitable UHMWPE liners exhibit stress at break (23° C.) up to 13,100 psi, with a strain at break (23° C.) of 360%, a tensile modulus (23° C.) as high as 140,000 psi, a storage modulus (23° C.) as high as 1300 psi, and a COF in air (23° C.) of 0.1 and a COF in saline (37° C.) of less than 0.1, values comparable to those of PTFE liners. Careful selection of materials based on these properties enables optimization for catheter trackability in a vasculature.

Advantageously, in some embodiments, the UHMWPE liners described herein do not require etching or any type of surface treatment to be effectively incorporated within a catheter assembly as described herein. Generally, the UHMWPE liners can be used without significant delamination between the liner and an adjacent layer without etching or surface treatment. However, the disclosure is not limited to unetched/unsurfaced-treated UHMWPE and in some embodiments, the disclosed liners can be surface treated and/or etched by various means known in the art.

In some embodiments, the UHMWPE liner is coated, such that it comprises a UHMWPE liner as described herein, which is partially or fully coated with a second resin, e.g., a tie resin that acts as a tie layer between the UHMWPE liner and an adjacent braid or jacket material in a corresponding catheter assembly. Any material known as a tie layer material within catheter constructions can reasonably be employed for this purpose. Suitable tie resins include, but are not limited to, copolymers of ethylene such as ethylene vinyl acetate (EVA) or functionalized polymers such as polyethylene resins grafted with maleic anhydride or acrylic acid. Other tie resins are known in the art.

### Reinforcement Layer

The catheter shaft can, in some embodiments, be reinforced with one or more materials that provide strength in the longitudinal direction (pushability), flexibility to maneuver through the vasculature, torque control from proximal to distal end, and/or crush resistance in the radial direction. This reinforcement can be applied, for example, as a braid, coil, or hybrid structure.

Braids are typically made of thin metal wires or high-strength polymer fibers woven into a mesh, adding mechanical strength and improving torque response. Braided layers are typically characterized by the dimensions of the cross-section of the wire or filament used and by the weave pattern and its density in picks per inch (ppi). Materials like stainless steel provide high tensile strength and excellent torque control, while Nitinol offers greater flexibility and kink resistance. For applications where flexibility is especially desirable over pushability, coils can be used in place of braids. In some embodiments, both coils and braids can be used.

### Outer Jacket

The outer jacket is the external layer of the catheter shaft, designed to provide a smooth, biocompatible surface for interaction with the body and to protect inner layers, enhancing the catheter's overall durability. Common materials for this layer include various grades of polyamides, polyurethanes and poly(ether block amide) s (PEBA) which can be tailored to specific mechanical properties such as softness, durability and flexibility. Certain lengths of the jacketing materials, such as the distal end or tip, can be further coated in some embodiments with hydrophilic and/or antimicrobial coatings to improve lubricity and reduce infection risks.

### Catheter Assembly

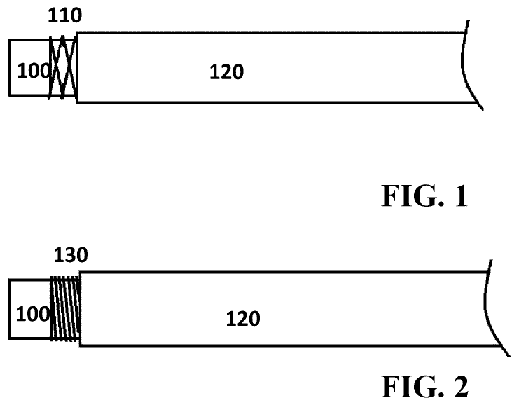

Catheter assemblies are provided herein, which comprise the disclosed UHMWPE liner (in coated or uncoated form), an optional reinforcement layer, and a jacketing layer. FIG. 1 shows such an assembly with a braid, and FIG. 2 shows such an assembly with a coil. Each component of a catheter shaft assembly plays a crucial role in the device's overall performance and effectiveness. While current materials and designs offer significant benefits, there are drawbacks that can be addressed to enhance patient outcomes and expand the range and effectiveness of possible medical applications.

Catheter assemblies comprising the disclosed liners can advantageously address one or more drawbacks of current catheters (e.g., comprising PTFE liners) while maintaining many of their desirable properties. In particular, the disclosed catheters can be sterilized by gamma or e-beam irradiation, thereby circumventing the issues associated with ETO sterilization. Liners based on polyethylene, as presently disclosed, can also make use of so-called tie resins to form covalent bonds with adjacent reinforcement or jacket layers, thereby reducing the propensity of the construction to delaminate.

The properties of the disclosed catheter assemblies can be similar to or better than existing catheters in terms of lubricity, pushability, and/or case of operation, as will be more fully disclosed hereinafter and as demonstrated by the Examples provided herein.

In some embodiments, the disclosed catheter assemblies exhibit comparable or improved physical properties as compared with comparable PTFE-lined catheter assemblies (but with the added advantage that they can be effectively sterilized via radiation). By “comparable PTFE-lined catheter assembly” or “comparable PTFE-lined catheter” is meant that these HDPE or PTFE liners would have approximately the same inner and outer diameters as the UHMWPE liner described herein.

In some embodiments, the disclosed catheter assemblies exhibit comparable or improved physical properties as compared with comparable HDPE-lined catheter assemblies. HDPE liners comprise polyethylene with typical molecular weight ranges of between 100,000 and 200,000 g/mol, which is easily processed by melt extrusion. HDPE liner-based catheters are described, e.g., in U.S. Pat. No. 7,815,625 to Stivland, which is incorporated herein by reference in its entirety. Tensile strength properties for HDPE are generally somewhat low for extruded tubes, e.g., as disclosed in International Patent Application Publication No. WO2013/149369, which is incorporated herein by reference, especially as compared with those for extruded PTFE tubes, e.g., as disclosed in U.S. Pat. No. 10,744,231, which is incorporated herein by reference. By “comparable PTFE-lined catheter assembly” or “comparable PTFE-lined catheter” is meant that these HDPE or PTFE liners would have approximately the same inner and outer diameters as the UHMWPE liner described herein.

Specifically, in some embodiments, the disclosed catheter assemblies can be characterized by their Guide Wire trackability, with lower Maximum Force and/or lower Work of Insertion for Guide Wires, e.g., than corresponding PTFE-lined catheter assemblies and/or HDPE-lined catheter assemblies. In some embodiments, a Maximum Force of Advancement of a 0.014″ guidewire is 40 mN or lower, 45 mN or lower, 50 mN or lower, 51 mN or lower, 52 mN or lower, 53 mN or lower, 54 mN or lower, or 55 mN or lower. In some embodiments, such values are at least 15% lower, at least 18% lower, at least 20% lower, at least 22% lower, or at least 25% lower than those of a comparable HDPE-lined catheter.

For example, as shown in Table 5 the UHMWPE catheter construction of Example 3 (composition described in Table 3) exhibits a Maximum Force of Advancement of a 0.014″ guidewire that is 51 mN, approximately 20% lower than a comparable HDPE-lined catheter (Example 3B), where the Maximum Force of Advancement was measured to be 64 mN. There was no significant difference in the Maximum Force of Advancement of a 0.014″ guidewire between the Example 3 UHMWPE catheter construction (51 mN) and the comparable PTFE catheter construction (Example 3A) (48 mN). Furthermore, the UHMWPE catheter construction of Example 3 exhibits a Maximum Work of Advancement of a 0.014″ guidewire that is 1564 mN·cm, which is more than 25% lower than a comparable HDPE-lined catheter (Example 3B), where the Maximum Work of Advancement was measured to be 2216 mN·cm. There was no significant difference in the Maximum Work of Advancement of a 0.014″ guidewire between the Example 3 UHMWPE catheter construction (1564 mN·cm) and the comparable PTFE catheter construction (Example 3A) (1486 mN·cm).

Thus, in the final constructions, the catheters of present disclosure can, in some embodiments, exhibit greater flexibility and/or greater lubricity and/or improved trackability than catheters constructed with HDPE liners and exhibit properties closer to those of catheters comprising PTFE liners.

The various tubes comprising components of the catheter can also be provided independently (i.e., not within a catheter) and can be employed in various applications.

### Production/Construction

It is generally understood that, e.g., due to its very high viscosity, processing UHMWPE to form a tube (e.g., suitable for use as a liner) is challenging. However, processing UHMWPE to form a liner for catheters can be accomplished by known methods, including, but not limited to, dispersion coating as described in U.S. Patent Application Publication No. 2024/0066186, which is incorporated herein by reference in its entirety and/or paste extrusion, as described in U.S. Patent Application Publication No. 2024/0066777, which is incorporated herein by reference in its entirety.

In some embodiments, the UHMWPE employed in these methods may be in a powder or pellet form. The UHMWPE may have an average particle diameter of less than 75 μm, or less than 70 μm, or less than 65 μm. The UHMWPE may have an average particle diameter of greater than 10 μm, or greater than 15 μm, or greater than 20 μm, or greater than 25 μm. In some embodiments, the UHMWPE may have an average particle diameter of from 40 to 75 μm, or from 50 to 70μ, or from 55 to 65 μm. In some embodiments, the UHMWPE may have an average particle diameter of from 10 to 50 μm, or from 15 to 45 μm, or from 20 to 40 μm, or from 25 to 30 μm.

One non-limiting method for construction of the catheter assemblies is carried out in the following manner. A UHMWPE liner as described herein is loaded onto a mandrel and the reinforcement layer is optionally introduced, e.g., the liner is braided or coiled with specified wire, for example, using a Steeger 16 carrier as braider and a Rothgreaves unit as coil winder. After the reinforcement layer is applied, the distal end can be trimmed and covered with a heat shrink tube (e.g., a PET heat shrink tube) to secure the optional reinforcement layer, e.g., braid or coil. The jacket tubing is then loaded onto the assembly, in some embodiments so that each durometer covers approximately half of the 3 ft long shaft. The assembly is then covered with another heat shrink material (e.g., FEP) and laminated, e.g., on a Machine Solutions 4-up, 200 cm long vertical laminator. Suitable laminator run conditions include, but are not limited to, a 500° F. nozzle temperature with a speed of 2.5 mm/s and air flow rate of 130 SCFH. The shrink tube is removed after cooling and the distal tip rounded for ease of tracking. The mandrel was then removed by stretching it, and the catheter shaft can be optionally sized, e.g., cut to the desired length (e.g., 36 inches overall length) to give the catheter assembly.

### Experimental

Embodiments of the present disclosure are more fully illustrated by the following examples, which are set forth to illustrate aspects of the present disclosure and are not to be construed as limiting thereof. Unless otherwise noted, all parts and percentages are by weight.

The individual components of a catheter can be tested in a variety of different ways to characterize their properties and assess their suitability for use in catheter constructions. An Instron 5965 dual column mechanical tester running Bluchill 3 v3.73.4823 operating software is used to determine the tensile properties of the tubing. The test is performed at a rate of 25.4 mm/min using a 1 kN load cell attached to pneumatic grips. Three test specimens are run, and average values of tensile properties such as Young's Modulus, Tensile Stress at Break and Elongation at Break are reported.

A TA instruments Q800 DMA with the film tension fixture can be used to determine the thermo-mechanical properties of tubing. The main property of interest is storage modulus (E′). A temperature scan is performed at a constant rate of 3° C./min while the test specimen is displaced at a constant amplitude of 15 μm with a fixed frequency tensile oscillation of 1 Hz. The resulting DMA data is imported into TA instruments TRIOS software v4.3, and the value of the storage modulus can be reported as a function of temperature.

A TA instruments Discovery Hybrid Rheometer (DHR-3) rheometer with the tribo-rheometer accessory is used to determine the tribological properties tubing. The main property of interest during this test is the coefficient of friction (COF). The specimens are prepared by attaching three tubing sections of 5 mm×16.5 mm each to the three teeth of the half-ring for use with a Ring-on-Plate tribo-rheometry fixture. The ring with mounted samples is then attached to the ring-on-plate upper-geometry holder and lowered to have the samples contact a mirror-finish stainless steel plate at the specified axial force. Tribological tests are performed at room temperature (23° C.) from sliding speeds of 750 μm/s to 7650 μm/s under an axial load of IN. Additional tribological tests can be performed in a saline bath and at elevated temperatures, such as 37° C. Minimum COF over the stated range in sliding speed is calculated by the TA instruments TRIOS software v4.3 and the average of three values is reported.

The performance of complete catheter shafts can be tested using interventional device testing equipment such as the IDTE 3000 from MSI which can measure and record device performance features such as track force, push efficiency, flexibility, and torqueability. The IDTE 3000 features a temperature-controlled water bath, adjustable pegboard tray that is submergible, proximal pneumatic roller system, proximal torque motor, and a dedicated PC that controls all operation.

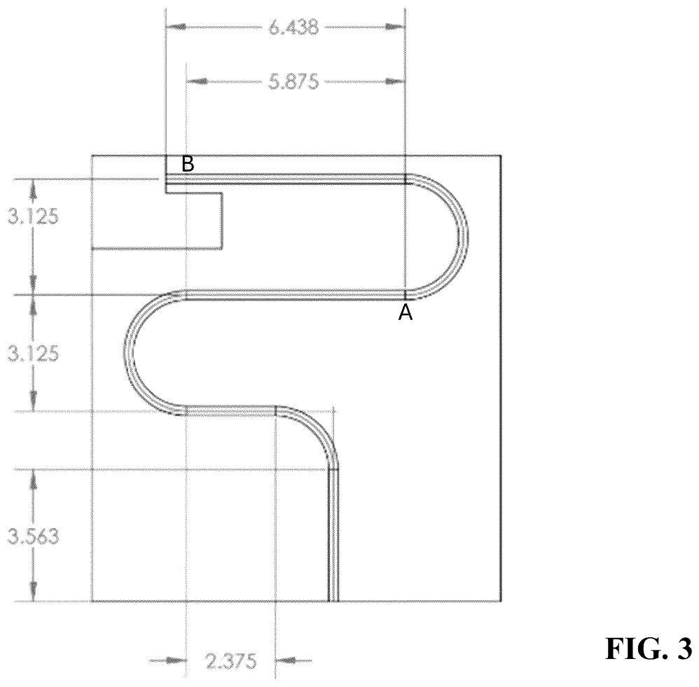

Testing is performed on the IDTE using the proximal pneumatic roller system. A standard setup for trackability consists of a product support tray, the proximal roller system, and a test track in an S-configuration (see FIG. 3) secured to the adjustable pegboard tray, wherein the pegboard tray is submerged in the water bath at 37° C. The proximal end of the test specimen is supported by the product tray, and the distal end feeds through the roller system and into the PFA tubing of the test track. The numbers shown in FIG. 3 refer to representative sizes (in inches), which are exemplary only and are not understood to be limiting of the setup.

A further test is tracking a guidewire through a lumen of the catheter to determine resistance to delivering a therapy through the catheter. In this case, the catheter is secured onto the test track and the proximal end is secured by a C-clamp on the roller system. A guidewire is laid flat on the product tray and the distal tip is fed through the roller system and into the lumen of the catheter until the tip is correctly positioned within the test track. This test method can be used to evaluate performance of a catheter liner by measuring the force required to insert and retract the guidewire through the catheter.

The following parameters are used to test trackability on the track shown in FIG. 3:
Roller pressure: 10-20 psi adjusted as necessary to prevent slippage or collapse of the test catheter wall at the proximal end.
Load cell: 3 Kg
Water temperature: 37° C.
Test speed: 100-150 cm/min

For catheter trackability, a 20 cm total test path is used extending from the proximal to the distal end of a PFA tube configured as the track according to the layout shown in FIG. 3. For guidewire trackability, a ViperWIRE Advance VPR-GW-14 Guidewire (0.014″) is used with the test catheter configured as the track according to the layout shown in FIG. 3.

The maximum force measured during the advancing portion of the test is recorded. The work performed to advance the catheter or guide wire is calculated using software such as OriginPro® to integrate the area beneath the plot of force (F) vs distance (x). The formula for calculating Work of Insertion (W) is:

$$W = {\int }_{A}^{B} F ( x ) dx , where A = initial position of the catheter and B = final position of the catheter .$$

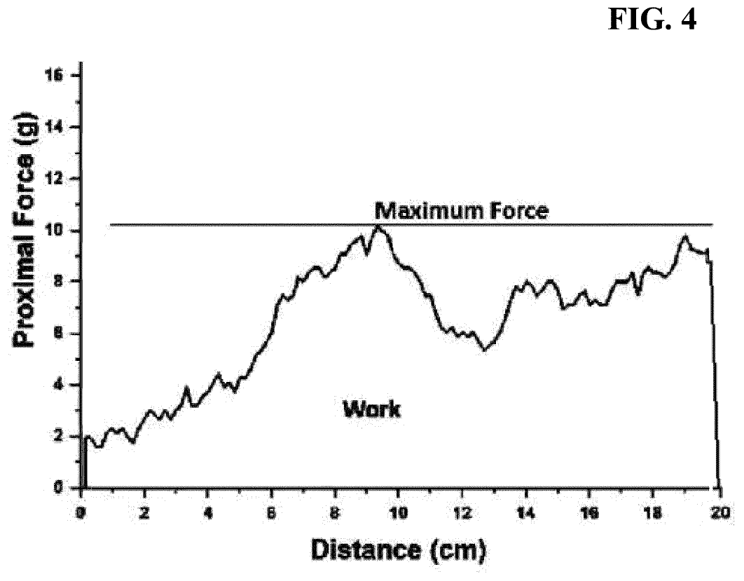

FIG. 4 gives an example of the salient features of a typical trackability curve.

### Examples

Table 1 summarizes the components utilized for the catheter builds.

TABLE 1
Components Used in The Catheter Builds

| Component | Material | Specifications |
|---|---|---|
| Liner 1 | PTFE | Zeus Streamliner SLW |
| | | etch; 0.0015″ wall |
| Liner 2 | HDPE | Bormed HE7541-PHS |
| Liner 3 | UHMWPE | Mipelon PM200; 0.0015″ |
| Wire 1 | SS | 304; flat; 0.001″ × 0.003″ |
| Wire 2 | SS | 304; flat; 0.001″ × 0.005″ |
| Wire 3 | SS | 304; flat; 0.002″ × 0.006″ |
| Wire 4 | SS | 304; round; 0.003″ |
| Wire 3 | Nitinol | Flat; 0.001″ × 0.005″ |
| PEBA 1 | Pebax | 55 Shore D Hardness |
| PEBA 2 | Pebax | 72 Shore D Hardness |

Liner 2 and Liner 3 were coated with a 0.0005″ tie layer that was melt extruded over the liners. The tie layer used for the polyethylene liners is a commercially available maleic anhydride grafted linear low-density polyethylene with a melt index of 1.5 (Rezilok Rx 101 available from Compounding Solutions, Lewiston, ME).

Physical properties of the liners and jackets are summarized in Table 2.

TABLE 2
Component Physical Properties

| | Liner 1 | Liner 2 | Liner 3 | PEBA 1 | PEBA 2 |
|---|---|---|---|---|---|
| Outer Diameter, in | 0.092 | 0.095 | 0.095 | 0.114 | 0.114 |
| Wall, in | 0.0012 | 0.0015 | 0.0019 | 0.0043 | 0.0044 |
| Stress @ Break | 13,100 | 8900 | 1760 | 12,100 | 8290 |
| (23° C.), psi | | | | | |
| Strain @ Break | 360 | 715 | 90 | 580 | 270 |
| (23° C.), % | | | | | |
| Tensile Modulus | 140,000 | 94,900 | 38,000 | 24,900 | 81,000 |
| (23° C.), psi | | | | | |
| Storage Modulus | 1300 | 520 | 311 | | |
| (23° C.), psi | | | | | |
| Storage Modulus | 760 | 380 | 240 | | |
| (37° C.), psi | | | | | |
| COF in air | 0.1 | 0.5 | 0.1 | | |
| (23° C.) | | | | | |
| COF in saline | <0.1 | 0.2 | <0.1 | | |
| (37° C.) | | | | | |

The tabulated components were used to build the catheter shafts shown below in Table 3. All braids were standard 70 ppi. Catheter shafts were 36 inches in overall length with the proximal half jacketed in Pebax 72D and the distal half jacketed in Pebax 55D.

TABLE 3
Catheter Constructions

| | Final | | | | | | | | | | |
|---|---|---|---|---|---|---|---|---|---|---|---|
| | Assembly Size | | Liner | | | Reinforcement | | | Jacket | | |
| | ID | OD | | ID | Wall | | Size | Type | | ID | Wall |
| Example # | inches | | Material | inches | | Material | inches | | Material | inches | |
| Example 1 | 0.013 | 0.029 | UHMWPE | 0.013 | 0.0015 | SS | 0.001 × | braid | 72 & 55 | 0.025 | 0.002 |
| | | | | | | | 0.003 | | | | |
| Comparative | 0.013 | 0.029 | PTFE | 0.013 | 0.0015 | SS | 0.001 × | braid | 72 & 55 | 0.025 | 0.002 |
| Example 1 | | | | | | | 0.003 | | | | |
| Example 2 | 0.013 | 0.027 | UHMWPE | 0.013 | 0.0015 | SS | 0.001 × | coil | 72 & 55 | 0.025 | 0.002 |
| | | | | | | | 0.003 | | | | |
| Comparative | 0.013 | 0.027 | PTFE | 0.013 | 0.0015 | SS | 0.001 × | coil | 72 & 55 | 0.025 | 0.002 |
| Example 2 | | | | | | | 0.003 | | | | |
| Example 3 | 0.030 | 0.046 | UHMWPE | 0.0305 | 0.002 | SS | 0.001 × | braid | 72 & 55 | 0.042 | 0.002 |
| | | | | | | | 0.003 | | | | |
| Comparative | 0.030 | 0.046 | PTFE | 0.030 | 0.0015 | SS | 0.001 × | braid | 72 & 55 | 0.042 | 0.002 |
| Example 3A | | | | | | | 0.003 | | | | |
| Comparative | 0.030 | 0.046 | HDPE | 0.030 | 0.002 | SS | 0.001 × | braid | 72 & 55 | 0.042 | 0.002 |
| Example 3B | | | | | | | 0.003 | | | | |
| Example 4 | 0.030 | 0.042 | UHMWPE | 0.0305 | 0.002 | none | N/A | N/A | 72 & 55 | 0.038 | 0.002 |
| Comparative | 0.030 | 0.042 | PTFE | 0.030 | 0.0015 | none | N/A | N/A | 72 & 55 | 0.038 | 0.002 |
| Example 4A | | | | | | | | | | | |
| Comparative | 0.030 | 0.042 | HDPE | 0.030 | 0.002 | none | N/A | N/A | 72 & 55 | 0.038 | 0.002 |
| Example 4B | | | | | | | | | | | |
| Example 5 | 0.071 | 0.095 | UHMWPE | 0.071 | 0.002 | SS | 0.001 × | braid | 72 & 55 | 0.085 | 0.005 |
| | | | | | | | 0.005 | | | | |
| Comparative | 0.071 | 0.095 | PTFE | 0.071 | 0.0015 | SS | 0.001 × | braid | 72 & 55 | 0.085 | 0.005 |
| Example 5 | | | | | | | 0.005 | | | | |
| Example 6 | 0.071 | 0.095 | UHMWPE | 0.071 | 0.002 | nitinol | 0.0005 × | braid | 72 & 55 | 0.085 | 0.005 |
| | | | | | | | 0.003 | | | | |
| Comparative | 0.071 | 0.095 | PTFE | 0.071 | 0.0015 | nitinol | 0.0005 × | braid | 72 & 55 | 0.085 | 0.005 |
| Example 6 | | | | | | | 0.003 | | | | |
| Example 7 | 0.071 | 0.101 | UHMWPE | 0.071 | 0.002 | SS | 0.003 | braid | 72 & 55 | 0.092 | 0.005 |
| | | | | | | | round | | | | |
| Comparative | 0.071 | 0.101 | PTFE | 0.071 | 0.0015 | SS | 0.003 | braid | 72 & 55 | 0.092 | 0.005 |
| Example 7 | | | | | | | round | | | | |
| Example 8 | 0.0915 | 0.118 | UHMWPE | 0.0915 | 0.0015 | SS | 0.002 × | braid | 72 & 55 | 0.108 | 0.005 |
| | | | | | | | 0.006 | | | | |
| Comparative | 0.0915 | 0.118 | PTFE | 0.0915 | 0.0015 | SS | 0.002 × | braid | 72 & 55 | 0.108 | 0.005 |
| Example 8 | | | | | | | 0.006 | | | | |
| Example 9 | 0.0915 | 0.114 | UHMWPE | 0.0915 | 0.0015 | SS | 0.002 × | coil | 72 & 55 | 0.104 | 0.005 |
| | | | | | | | 0.006 | | | | |
| Comparative | 0.0915 | 0.114 | PTFE | 0.0915 | 0.0015 | SS | 0.002 × | coil | 72 & 55 | 0.104 | 0.005 |
| Example 9A | | | | | | | 0.006 | | | | |
| Comparative | 0.0915 | 0.114 | HDPE | 0.0915 | 0.0015 | SS | 0.002 × | coil | 72 & 55 | 0.104 | 0.005 |
| Example 9B | | | | | | | 0.006 | | | | |
| Example 10 | 0.387 | 0.4275 | UHMWPE | 0.3856 | 0.0035 | SS | 0.002 × | braid | 72 & 55 | 0.4075 | 0.010 |
| | | | | | | | 0.006 | | | | |
| Comparative | 0.387 | 0.4275 | PTFE | 0.387 | 0.00375 | SS | 0.002 × | braid | 72 & 55 | 0.4075 | 0.010 |
| Example 10 | | | | | | | 0.006 | | | | |

Test results for catheter trackability are summarized in Table 4. Test results for guidewire trackability inside the catheter lumen are summarized in Table 5.

TABLE 4
Trackability Data for the Catheters of Table 3

| | Maximum | Work of |
|---|---|---|
| | Force, | Advancement, |
| Example # | mN | mN · cm |
| Example 1 | 47 | 647 |
| Comp. Example 1 | 42 | 588 |
| Example 2 | | |
| Comp. Example 2 | | |
| Example 3 | 49 | 637 |
| Comp. Example 3A | 77 | 785 |
| Comp. Example 3B | 57 | 706 |
| Example 4 | NA | NA |
| Comp. Example 4A | NA | NA |
| Comp. Example 4B | NA | NA |
| Example 5 | 373 | 4874 |
| Comp. Example 5 | 710 | 7183 |
| Example 6 | 372 | 5055 |
| Comp. Example 6 | 467 | 5634 |
| Example 7 | 374 | 4923 |
| Comp. Example 7 | 409 | 5188 |
| Example 8 | 1275 | 13700 |
| Comp. Example 8 | 1811 | 19540 |
| Example 9 | 1111 | 11080 |
| Comp. Example 9A | 1160 | 12480 |
| Comp. Example 9B | 1640 | 14320 |
| Example 10 | | |
| Comp. Example 10 | | |

TABLE 5
Trackability Data for a Guide Wire
through the Catheters of Table 3

| | Maximum | Work of |
|---|---|---|
| | Force, | Advancement, |
| Example # | mN | mN · cm |
| Example 1 | | |
| Comp. Example 1 | | |
| Example 2 | | |
| Comp. Example 2 | | |
| Example 3 | 51 | 1564 |
| Comp. Example 3A | 48 | 1486 |
| Comp. Example 3B | 64 | 2216 |
| Example 4 | 99 | 3903 |
| Comp. Example 4A | 71 | 2354 |
| Comp. Example 4B | 96 | 3795 |
| Example 5 | 44 | 1976 |
| Comp. Example 5 | 23 | 2452 |
| Example 6 | 57 | 2133 |
| Comp. Example 6 | 51 | 1937 |
| Example 7 | 52 | 1804 |
| Comp. Example 7 | 45 | 1667 |
| Example 8 | 50 | 1971 |
| Comp. Example 8 | 45 | 1672 |
| Example 9 | 60 | 2373 |
| Comp. Example 9A | 54 | 2099 |
| Comp. Example 9B | 58 | 2491 |
| Example 10 | | |
| Comp. Example 10 | | |

Catheter trackability is a measure of the ease with which a catheter can be advanced through the vasculature. The catheters of the invention comprising UHMWPE liners (Examples 3 and 9) are noted to give lower values of Maximum Force and Work of Advancement relative to comparable catheters constructed with HDPE liners (Comp. Examples 3B and 9B, respectively). Hence, the catheters of the invention would be expected to navigate the vasculature with greater ease.

Guide Wire trackability is a sensitive measure of the lubricity of the liner material in the final catheter construction. Without the presence of a coil or braid the Maximum Force and Work of Insertion for both UHMWPE and HDPE liners is about the same (compare Example 4 and Comp. Example 4B). Surprisingly, catheters within the scope of the disclosure (e.g., Example 3) exhibit lower Maximum Force by 16% and lower Work of Insertion for Guide Wires by 41% compared to catheters with HDPE liners (Comp. Example 3B) for catheters with an OD of 0.046″. For larger catheters with an OD of 0.114″, the trend is still observed with Maximum Force being about the same but Work of Advancement being lower for the UHMWPE catheter by about 5%. Thus, in the final constructions, the catheters of the current disclosure can exhibit greater flexibility and greater lubricity than catheters constructed with HDPE liners and exhibit properties closer to those of catheters comprising PTFE liners.

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

1. A catheter comprising an Ultra High Molecular Weight Poly(Ethylene) (“UHMWPE”) liner, an optional reinforcement layer, and a jacket,
wherein the UHMWPE liner has an inner diameter (“ID”) of 0.013″ to 0.400″, and
wherein the catheter exhibits both of:
a) a Maximum Work of Advancement of 12,000 mN·cm or less; and
b) a Maximum Work of Advancement of a 0.014″ guidewire of 2500 mN·cm or less.

2. The catheter of claim 1, further comprising a tie layer overlying at least a portion of the UHMWPE liner.

3. The catheter of claim 2, wherein the tie layer comprises a functionalized polyethylene.

4. The catheter of claim 3, wherein the functionalized polyethylene comprises maleic anhydride grafted polyethylene.

5. The catheter of claim 1, comprising the reinforcement layer.

6. The catheter of claim 5, wherein the reinforcement layer is selected from the group consisting of a coil, a braid, or a combination thereof.

7. The catheter of claim 5, wherein the reinforcement layer comprises a metallic or polymeric material.

8. The catheter liner of claim 1, wherein the UHMWPE liner comprises UHMWPE comprising at least 85% polymerized ethylene-derived monomer units.

9. The catheter liner of claim 8, wherein the UHMWPE further comprises one or more α-olefins.

10. The catheter liner of claim 9, wherein the one or more α-olefins are selected from the group consisting of 1-butene, 1-pentene, 1-hexene, 1-octene, 1-decene, 1-dodecene, 4-methyl-1-pentene, 3-methyl-1-pentene, and combinations thereof.

11. The catheter of claim 1, wherein the UHMWPE liner consists essentially of UHMWPE.

12. The catheter of claim 1, wherein the UHMWPE liner comprises one or more particulate fillers.

13. The catheter of claim 1, wherein the UHMWPE liner consists essentially of UHMWPE and one or more particulate fillers.

14. The catheter of claim 1, wherein the catheter exhibits a Maximum Force of Advancement of a 0.014″ guidewire that is at least about 20% lower than a Maximum Force of Advancement of a 0.014″ guidewire of a comparable HDPE-lined catheter.

15. The catheter of claim 1, wherein the catheter exhibits a Work of Advancement of a 0.014″ guidewire that is at least about 25% lower than a Work of Advancement of a 0.014″ guidewire of a comparable HDPE-lined catheter.

16. The catheter of claim 1, wherein the catheter exhibits a Work of Advancement of a 0.014″ guidewire that is within about 20% of a Work of Advancement of a 0.014″ guidewire of a comparable PTFE-lined catheter.

17. The catheter of claim 1, wherein the catheter exhibits a maximum Force of Advancement of 1200 mN or less.

18. The catheter of claim 1, wherein the catheter exhibits a maximum Work of Advancement of 12,000 mN·cm or less.

19. The catheter of claim 1, wherein the catheter exhibits a maximum Force of Advancement of a 0.014″ guidewire of 60 mN or less.

20. The catheter of claim 1, wherein the catheter exhibits a maximum Work of Advancement of a 0.014″ guidewire of 2500 mN·cm or less.

21. The catheter of claim 1, wherein the UHMWPE liner has a wall thickness of about 0.001 to 0.002 inches.

22. The catheter of claim 1, wherein the UHMWPE liner has been formed by a dip-coating process.

23. (canceled)

24. The catheter of claim 1, wherein the catheter has an outer diameter (OD) of 0.027″ to 0.4275″.

25. The catheter of claim 1, wherein the catheter consists essentially of the UHMWPE liner, the optional reinforcement layer, and the jacket.
